(19) United States
(12) Patent Application Publication
(10) Pub. No.: US 2006/0106369 A1
(43) Pub. Date: May 18, 2006

(54) Haptic interface for force reflection in manipulation tasks

(76) Inventors: Jaydev P. Desai, Wallingford, PA (US); Gregory Tholey, Holland, PA (US)

Correspondence Address: KNOBLE, YOSHIDA & DUNLEAVY, EIGHT PENN CENTER, SUITE 1350, 1628 JOHN F KENNEDY BLVD, PHILADELPHIA, PA, 19103, US

(21) Appl. No.: 11/271,115

(22) Filed: Nov. 10, 2005

Related U.S. Application Data
(60) Provisional application No. 60/627,380, filed on Nov. 12, 2004.

(51) Int. Cl. A61B 17/00

(52) U.S. Cl. 606/1

(57) ABSTRACT

A haptic interface was designed and developed for use in manipulation tasks. The mechanism consists of a closed kinematic chain. The force feedback mechanism consists of three degrees of spatial force feedback (X, Y and Z directions) and 1 degree of grasping/parting (increasing the distance between two or more points) force feedback. The three degrees of spatial force feedback are comprised of three independently actuated prismatic joints along orthogonal coordinate axes. The one degree of grasping/parting force feedback consists of a two thimbles with rotary motion for grasping and parting tasks using one's fingers. This device also provides a net of three additional passive joints for a total of 7 degrees-of-freedom.

This application claims the benefit of U.S. provisional patent application no. 60/627,380, filed on Nov. 12, 2004, under 35 U.S.C. §119(e).

### BACKGROUND OF THE INVENTION

1. Field of the Invention

The present invention relates generally to haptic interfaces that provide force reflection and more specifically to robot-assisted surgical devices.

2. Description of the Prior Art

Robot-assisted surgical systems have led to significant improvements within the medical field. These systems could lead to better performance in minimally invasive surgery (MIS), thereby reducing patient trauma, recovery time, and lowering health care costs, to name a few. While these systems have the above advantages, they also have shortcomings, such as high cost, inability to use qualitative information, and lack of haptic feedback [1]. Several researchers have already proposed solutions for the lack of haptic feedback in robot-assisted surgery through the development of surgical tools with force-sensing capabilities [2-15]. These solutions have incorporated force sensors on the tool with direct or indirect measurements of the tool-tissue interaction forces. This represents half of the solution to the problem of lack of haptic feedback in surgery. The other half of the solution requires the accurate reflection of the measured tool-tissue interaction forces back to the surgeon through a haptic interface.

Many researchers have proposed and developed different types of haptic devices for various applications. Massie and Salisbury [16] developed the Personal Haptic Interface Mechanism (PHANToM™), which is commercially available and used for many different applications. Haptic devices have been developed using serial and parallel mechanisms [17-25]. Serial mechanisms have the advantage of a large workspace while parallel mechanisms have the advantage that they have a compact footprint and provide high force output. Still others have developed haptic glove or exoskeleton devices that offer advantages such as a large workspace and a relatively large number of degrees of freedom [26-28]. A few researchers have also developed laparoscopic and surgical haptic mechanisms that can be used for MIS and robot-assisted MIS [29-33].

A “haptic interface device” provides a haptic sensation (haptic display) to a user of the haptic interface device in response to the user's interaction with an environment with which the haptic interface device is associated. “Haptic” refers to the sense of touch: haptic interface display devices thus produce sensations associated with the sense of touch, such as texture, force (e.g., frictional force, magnetic repulsion or attraction), vibration, mass, density, viscosity, temperature, moisture, or some combination of such sensations. Haptic interface devices can be embodied in a variety of different apparatus, such as, for example, apparatus for conveying force and/or vibrotactile sensation (e.g., a stylus, a movable arm, a wheel, a dial, a roller, a slider or a vibratory surface), apparatus for conveying thermal sensation (e.g., a thermally-controlled surface or air volume), and apparatus for conveying the sensation of moisture (e.g., a moisture-controlled surface or air volume).

Haptic interface devices can be used in a wide variety of applications. For example, some joysticks and computer mice incorporate force feedback to provide a haptic display to a user of the joystick or mouse. Some paging devices are adapted to vibrate when a paging signal is received. Some toys produce vibrations as part of the interaction with the toy. These examples give an indication of the range of applications for which haptic interfaces can be used.

The two different forms of human haptic perception that haptic interface systems attempt to replicate are tactile and kinesthetic. The human tactile system consists of nerve endings in the skin which respond to pressure, warmth, cold, pain, vibration and itch. The tactile system allows humans to sense local geometry, texture, and thermal properties from static contact. The kinesthetic system refers to the collection of receptors in the muscles, tendons, and joints which allow perception of the motion and forces upon a human's limbs. In order to accurately replicate the forces experienced by humans in the real world, haptic interface systems attempt to model the shape, surface compliance and texture of objects.

In typical multi-degrees of freedom apparatus that include force feedback, there are several disadvantages. Since actuators which supply force feedback tend to be heavier and larger than sensors, they may create inertial constraints if added to existing devices. There is also the problem of coupled actuators. In a typical force feedback device, a serial chain of links and actuators is implemented to achieve multiple degrees of freedom in a desired object positioned at the end of the chain, i.e., each actuator is coupled to the previous actuator. The user who manipulates the object must carry the inertia of all of the subsequent actuators and links except for the first actuator in the chain, which is grounded. While it is possible to ground all of the actuators in a serial chain by using a complex transmission of cables or belts, the end result could be a low stiffness, high friction, high damping transmission which corrupts the bandwidth of the system, providing the user with an unresponsive and inaccurate interface. These types of interfaces also introduce tactile “noise” to the user through friction and compliance in signal transmission and limit the degree of sensitivity conveyed to the user through the actuators of the device.

Force reflecting hand controllers for tele-operation are well known. Units that reflect the force sensed by a remote manipulator are disclosed in U.S. Pat. No. 4,837,734 to Ichikawa et al., U.S. Pat. No. 4,853,874 to Iwamoto et al., U.S. Pat. No. 4,888,538 to Dimitrov et al., U.S. Pat. Nos. 4,893,981 and 5,018,922 to Yoshinada et al., U.S. Pat. No. 4,942,538 to Yuan et al., U.S. Pat. No. 5,004,391 to Burdea, and U.S. Pat. No. 5,053,975 to Tsuchihashi et al. These units use force feedback, usually applied through an electric motor/gear drive, to present forces sensed by a remote manipulator to a user. Other existing devices may provide force feedback to a user. In U.S. Pat. No. 5,184,319, an interface is described which provides force and texture information to a user of a computer system. The interface consists of a glove or “exoskeleton” which is worn over the user's appendages, such as fingers, arms, or body. Forces can be applied to the user's appendages using tendon assemblies and actuators controlled by a computer system to simulate force and textual feedback. However, this system as described is not easily applicable to simulation environments such as those mentioned above where an object is referenced in 3D space and force feedback is applied to the object. As the forces are applied to the user with reference to the body of the user; the absolute location of the user's appendages are not easily calculated. In addition, such exoskeleton devices can be cumbersome or even dangerous to the user if extensive devices are worn over the user's appendages. Furthermore, the devices disclosed are complex mechanisms in which many actuators must be used to provide force feedback to the user.

U.S. Pat. No. 6,801,008 describes a method and system for providing a tactile virtual reality in response to user position and orientation. This system effects and controls the superposition of translational displacement with force application and angular displacement with torque, thus providing arbitrary, programmed application of forces, torques, and displacements to the user in any direction, thereby allowing the device to be controlled by, and to control, external simulations or models as well as physically remote devices. The device may also locally simulate virtual force fields generated from interaction with virtual surfaces and/or boundaries, can provide software programmed position, velocity, force, and acceleration limit stops, and can dynamically shift, rotate, or scale these virtual objects.

U.S. Pat. No. 6,723,106, describes a surgical manipulator that includes a mechanism with a plurality of arms. The manipulator enhances the dexterity of the operator while reducing the fatigue to the user. This surgical manipulator has the disadvantage of being bulky in size and limited in the number of haptic interfaces available to the user.

U.S. Pat. No. 6,369,834, describes a method and apparatus for determining forces to be applied to a user interacting with virtual objects in a virtual reality computer environment. Specifically, a method and apparatus for determining forces to be applied to a user through a haptic interface is described.

U.S. Pat. No. 6,088,020, describes a haptic device that extends the number of active degrees of freedom of haptic interface provided to the user. The apparatus described, has a 4-degrees of freedom gimbal, the shaft of the tool handle, whose tip is controlled by another 3 spatial degrees of freedom haptic device. The shaft of the tool slides and rotates in a sleeve bearing or collar which is mounted in a 2 degrees of freedom gimbal. The gimbal is rigidly connected to a 2 degrees of freedom parallel planar manipulator, with both degrees of freedom of the planar manipulator being powered by actuators used to generate the requisite haptic forces. The use of this device provides users with a 5-degrees of freedom device, through which they can feel forces and moments, instead of only point forces which are generated by 3-degrees of freedom devices. This is useful when performing simulations where a portion of the tool distant from the tip may contact an obstruction instead of just the tip.

All of these haptic devices have their own distinct advantages for use in robot-assisted surgery; however, they also have many disadvantages. Serial mechanisms, such as the PHANToM™, lack a sufficient force feedback capability without adding significant weight and do not have a grasping/parting interface capable of providing force feedback. Parallel mechanisms overcome the force issue, however, they have a smaller workspace and also lack a grasping/parting interface. Glove-type haptic feedback devices have also been explored as they have a large workspace and many degrees of freedom for grasping and/or parting. Laparoscopic haptic mechanisms have incorporated the grasping/parting interface but can only reflect laparoscopically based MIS procedures. Therefore, a need exists for the development of a surgical haptic interface that can reflect forces for any type of robot-assisted surgical procedure.

### SUMMARY OF THE INVENTION

Thus, a surgical haptic interface that can reflect forces for any type of robotically assisted surgical procedure has been developed. A haptic interface with multiple degrees of freedom position feedback which also provides force feedback has been developed. The mechanism may provide force feedback along three orthogonal axes and for a grasping/parting direction. This interface can also be used for a variety of other applications such as automotive industry, gaming industry, and as a rehabilitation aid for people with finger, hand, and/or forearm injuries, etc.

### BRIEF DESCRIPTION OF THE DRAWINGS

FIG. 1 is a schematic three-dimensional view of a haptic interface in accordance with the invention.

FIG. 2 is a schematic three-dimensional rear view of the haptic interface of FIG. 1.

FIG. 3 is a schematic of a front view of the haptic interface of FIG. 1.

FIG. 4 is a schematic of a side view of the haptic interface of FIG. 1.

FIG. 5 is an expanded schematic of a hand/forearm rest for use in a haptic interface in accordance with the present invention.

FIG. 6 is an expanded schematic (top) view of an exemplary grasping/parting assembly.

FIG. 7 is an expanded schematic view of a universal joint between the grasping/parting assembly and a spatial force feedback mechanism.

FIG. 8 is a schematic of a three degrees-of-freedom spatial force feedback mechanism.

FIG. 9 is an alternative embodiment of the haptic interface in accordance with the invention.

FIG. 10 is an expanded schematic (top) view of an alternative embodiment of a grasping/parting assembly.

### DETAILED DESCRIPTION OF THE INVENTION

Most haptic interfaces do not have an adequate range of force feedback capability for a given workspace volume. Additionally, some haptic interfaces are too bulky, particularly if higher ranges of force feedback are present within the system. Other haptic feedback systems have coupled motor torques to provide the necessary feedback at the tip. Lastly, some haptic feedback systems are ergonomically unfriendly to the user.

The present invention overcomes these disadvantages by providing four independently actuated joints for force feedback while at the same time netting three additional passive joints to provide better maneuverability. This provides for a better ergonomic design that is more user friendly. The present invention also provides for a larger force feedback capability as well as providing for a larger workspace for the given range of the force feedback capability. Additionally, the present invention has a single independent motor for each of the X, Y and Z direction force feedback axes and the grasping/parting assembly. Finally, the present invention provides for an intuitive haptic interface for spatial manipulation.

The present invention is designed as a non-portable haptic interface, which allows the use of higher power actuators, as the user does not carry the complete weight of the spatial (X, Y, and Z directions) actuators but would feel some inertia of the overall device. The higher-powered actuator carried by the user for grasping and parting tasks is adequately counterbalanced to reduce fatigue. The provision of a single independent motor for each of the X, Y and Z direction force feedback axes combined with a single independent motor for the grasping and parting forces represents an improvement over the prior art.

“Actuator” as used in this specification refers to a unit that is either a motor, or otherwise exerts a force. The actuator is often equipped with an encoder, although, it need not be. A high-resolution encoder suitable for use with an actuator provides 2,000 counts per rotation, and is available from Hewlett-Packard of Palo Alto, Calif. under model number 5310.

Briefly, an actuator has a body portion and an axle upon which is mounted a capstan. If current is provided to the actuator, the capstan spins on the axis relative to the body portion. A mounting bracket rotationally fixes the body portion, so the capstan rotates relative to an axis and the body portion remains fixed. A cable is wrapped around the capstan and is anchored at either end. The cable is routed such that when the capstan rotates, it pulls the cable around it, thus causing movement of an axis.

A capstan drive mechanism is advantageously used in the present invention to provide transmission of forces and mechanical advantage between an actuator and an object without introducing substantial compliance, friction, or backlash to the system. A capstan drive provides increased stiffness, so that forces are transmitted with negligible stretch and compression of the components. The amount of friction is also reduced with a capstan drive mechanism so that substantially “noiseless” tactile signals can be provided to the user. In addition, the amount of backlash contributed by a capstan drive is also negligible. “Backlash” is the amount of play that occurs between two coupled rotating objects in a gear or pulley system.

Two gears, belts, or other types of drive mechanisms could also be used in place of the capstan drive mechanism in alternate embodiments to transmit forces between actuator and member. However, gears and the like typically introduce some backlash in the system. In addition, a user might be able to feel the interlocking and grinding of gear teeth during rotation of gears when manipulating an object. Generally, the rotation in a capstan drive mechanism is much less noticeable than the rotation of gears.

Cables have a finite minimum pulley radius around which they may travel without creating friction and being significantly fatigued. For instance, for cables sold by Sava Corporation under trade designation ST-2032, suitable for use with the embodiment described above, 0.028 inches (0.71 mm) in diameter, the minimum radius is 0.2 in. (5.08 mm). Transmissions should avoid excessive free lengths of cables over long spans. Long lengths of free cables introduce compliance into the transmission. Further, pretensioned lengths of cables act as energy sources, which can lead to unwanted resonances at certain frequencies.

Finally, it is often helpful to add a spiral groove to capstans. This insures that the cable travels in the same manner each time and that wraps of the cable do not scrape each other. This groove also effectively increases the friction coefficient between the cable and capstan, as well as also reducing the fatigue in the cable, both of which are desirable. Other types of durable cables, cords, wire, etc. can be used as well.

The present invention provides an interface between motion of an object with an electrical system that includes a sensor, such as a digital encoder, that detects movement of an object along a degree of freedom. The sensor is preferably coupled to the object. The sensor has a sensing resolution, and presumably an amount of play less than the sensing resolution exists between the sensor and the object. More preferably, an amount of play that is an order of magnitude less than the sensing resolution, or a negligible amount of play, exists between the sensor and object.

The apparatus also includes an actuator assembly that includes an actuator coupled to the object to transmit a force to the object along the degree of freedom. The actuator may be an electromechanical passive resistance element, such as a magnetic particle brake. The actuator assembly may also include a play mechanism that is coupled to the actuator for providing a desired amount of play between the actuator and the object along the degree of freedom. The desired amount of play is greater than the sensing resolution of the sensor so that the sensor can detect the play. Such desired play can include torsion flex (compliance) or rotary backlash. When the play is provided as rotary backlash, the actuator is preferably coupled to a coupling having a keyed bore which is smaller than a keyed shaft that is received by the keyed bore. The actuator and the sensor provide an electromechanical interface between the object and the electrical system.

In an alternative embodiment, the apparatus may include a sensor and braking mechanism for sensing and providing force feedback along a second degree of freedom provided by a grasping/parting mechanism. The braking mechanism includes an actuator and coupling to provide the desired amount of play. A capstan drive mechanism is coupled between the actuator and the grasping/parting mechanism. The capstan drive mechanism transmits the force generated by the actuator to the grasping/parting mechanism and transmits forces applied to the grasping/parting mechanism by a user to the sensor. A linear axis member can also be coupled to the grasping/parting mechanism at the intersection of the two axes of rotation. The object is coupled to the linear axis member and the linear axis member and object can be translated along a third axis in a third degree of freedom. A third degree of freedom actuator can be used to create a drag along the third degree of freedom and to sense translation of the linear axis member. Sensors can also be included for sensing positions of said object along fourth, fifth, sixth and seventh degrees of freedom. The object can be a surgical tool, a stylus, an industrial tool, a joystick, or similar articles.

An electromechanical input/output device according to the present invention may include an object capable of moving along at least one degree of freedom. A sensor senses movement along the provided degrees of freedom and provides an electrical signal from this movement. An electromechanical brake mechanism applies a resistive force to the object along said at least one degree of freedom and is responsive to a braking signal, and a play mechanism couples the brake mechanism to the object. The sensor can detect movements of the object along the degree of freedom when the brake mechanism is engaged due to the play mechanism. Preferably, the sensor is coupled to the object and detects rotational movement of the object. The play mechanism includes a coupling rigidly coupled to the object and non-rigidly coupled to the brake mechanism. The object is capable of being moved along the degree of freedom by a user who is grasping the object. A computer system preferably provides the braking signal to the brake mechanism and receives the electrical signal from the sensor.

In an alternative embodiment of the present invention, a system for controlling an electromechanical interface apparatus manipulated by a user includes a digital computer system for receiving an input control signal and for providing an output control signal which updates a process in response to the input control signal. A passive actuator for receiving the output control signal provides a resistive force along a degree of freedom to an object coupled to the passive actuator. The object is preferably grasped and moved by the user. The resistive force is based on information in the output control signal and resists a force applied to the object by the user along the degree of freedom. A sensor detects motion of the object and outputs the input control signal including information representative of the position and motion of the object to the digital computer system. Preferably, the digital computer updates a simulation process in response to the input control signal and displays a simulation to the user on a display screen. A play mechanism preferably provides a desired amount of play between the actuator and the object, the desired amount of play being greater than a sensing resolution of the sensor. A serial interface or additional hardware can output the output control signal from the computer system and can receive the input control signal to the computer system. A digital to analog converter can receive the output control signal, convert the output control signal to an analog control signal, and output the analog control signal to the passive actuator. Finally, a microprocessor can provide the output control signal from the serial interface or any additional hardware to the digital to analog converter and can receive the input control signal from the sensor.

A method for interfacing motion of an object with an electrical system includes the steps of defining an origin in 3-dimensional space and providing a grasping/parting mechanism movable relative to the origin such that an object engaged with the grasping/parting mechanism has a degree of freedom. Positions of the object along the degree of freedom are sensed with a sensor such that play less than the sensing resolution of the sensor is allowed between the sensor and the object. A drag is created from a brake along the degree of freedom, and a desired amount of play greater than or equal to the sensing resolution of the sensor is allowed between the actuator and the object. Output electrical signals from the electrical system are converted into movement of the object and movement of the object is converted into electrical signals input to the electrical system. The play preferably includes rotary backlash and/or torsion flex.

In another method for controlling an interface apparatus according to the present invention, steps include sensing the current position of an object coupled to an interface apparatus and determining the difference between the current position of the object and a previous position of the object. A magnitude of a resistive force to be applied to the object is determined; this magnitude is based at least in part on the difference between the current position and the previous position. A control signal is provided to a passive actuator to transmit a resistive force having the determined magnitude to the object. The above steps are repeated as the user moves the object. The current position of the object is preferably sensed even when the object is locked into a position by the passive actuator. Preferably, a damping constant is determined which is multiplied by the difference to determine the magnitude of the resistive force. The above steps can be implemented for a plurality of sensor and passive actuator pairs.

The interface of the present invention may include a system having an actuator and a sensor. The actuator may be a passive actuator, such as magnetic particle brakes, that require less power and slower control signals than active actuators. A desired amount of play, such as backlash or compliance, may be provided between the actuator and an interfaced object so that a controlling computer can determine the direction that a user moves the object, even when the passive actuators are holding the object stationary. In addition, the user preferably cannot feel the play in the system. The actuator and sensor system can be used on a variety of mechanical interfaces providing one to six degrees of freedom and can also be used with capstan drive mechanisms so that the desired play is substantially the only play introduced to the interface system. These improvements allow a computer system to have more complete and accurate control over a low-cost interface providing realistic force feedback.

A haptic interface device in accordance with the invention can be used in a variety of applications, such as, for example, robot-assisted surgery, telesurgery, and in industrial tools.

A haptic interface device in accordance with the invention may be used in a similar manner for applications in which the user interacts with a volumetric data set (i.e., a three-dimensional representation of something such as a physical object). A haptic interface device can be used to navigate in three-dimensions around the space represented by the volumetric data set and provide haptic sensations (which can also be in three-dimensions) corresponding to characteristics of part of the volumetric data set with which the user is interacting. For example, there are a variety of medical simulation applications in which a volumetric data set is used to model some part or all of the human body. A haptic interface device can be used to navigate about the modeled part of the body and provide haptic sensations (such as compliance, inertia or texture) corresponding to characteristics (such as change in thickness or hardness of soft tissue and/or organs) of the modeled part of the body with which the user is interacting. The invention enables the resolution of such a haptic interface device to be varied by the user as the user navigates about the modeled part of the body, so that, if the user encounters something of interest at a particular location, the user can begin to navigate around that location at an increased level of granularity, thus enabling heightened scrutiny of that part of the modeled part of the body. Since the haptic interface device can be moved in any of three dimensions to navigate the volumetric data set, it may be necessary to provide for resolution control that is effected other than by applying force and/or motion in one of those directions: for example, the haptic interface device can include a pushbutton or a squeezable handle that, when depressed, changes the haptic resolution at a predetermined rate.

Actuators may be linear current control motors, such as DC servo motors. These motors preferably receive current signals to control the direction and torque (consequently the force output) that is produced on a shaft; the control signals for the motor are produced by computer interface on the control buses. The motors may include brakes which allow the rotation of the shaft to be halted in a short span of time. A suitable actuator and sensor pair for the present invention including both an optical encoder and current controlled motor is a 20 W basket wound servo motor manufactured by Maxon of Burlingame, Calif.

In alternate embodiments, other types of motors can be used, such as a stepper motor controlled with pulse width modulation of an applied voltage, or pneumatic motors. However, the present invention is much more suited to the use of linear current controlled motors. This is because voltage pulse width modulation or stepper motor control involves the use of steps or pulses which can be felt as “noise” by the user. Such noise corrupts the virtual simulation. Linear current control is smoother and thus more appropriate for the present invention.

Magnetic particle brakes or friction brakes can be used in addition to or instead of a motor to generate a passive resistance or friction in a degree of motion. An alternate preferred embodiment only including passive actuators may not be as realistic as an embodiment including motors; however, the passive actuators are typically safer for a user since the user does not have to fight generated forces.

In other embodiments, all or some of the actuator and sensor pairs can include only sensors to provide an apparatus without force feedback along designated degrees of freedom. Similarly, all or some of the actuator and sensor pairs can be implemented as actuators without sensors to provide only force feedback.

Digital sensors provide signals to a computer relating the position of the user object in 3D space. In alternative embodiments, sensors are relative optical encoders, which are electro-optical devices that respond to an axis rotation by producing two phase-related signals. In this embodiment, a sensor interface circuit, which may be a single chip, receives the signals from digital sensors and converts the two signals from each sensor into another pair of clock signals, which drive a bi-directional binary counter. The output of the binary counter is received by computer as a binary number representing the angular position of the encoded shaft. Such circuits, or equivalent circuits, are well known to those skilled in the art; for example, the Quadrature Chip from Hewlett Packard, Calif. performs the functions described above.

Analog sensors may be used instead of digital sensors for all or some of the sensors of the present invention. Analog sensors provide an analog signal representative of the position of the user object in a particular degree of motion. Analog to digital converter (ADC) converts the analog signal to a digital signal that is received and interpreted by computer, as is well known to those skilled in the art.

Regardless of the sensor type, the sensor must be able to detect directional rotational movements about the various axes and transmit that to the user. The operation of such sensors are well-known to those skilled in the art.

Ideally the sensor has a sensing resolution, which is the smallest change in rotational position of axis that the sensor can detect. For example, an optical encoder of the described embodiment may be able to detect on the order of about 3600 equally-spaced “pulses” (described below) per revolution of axis, which is about 10 detected pulses per degree of rotational movement. Thus, the sensing resolution of this sensor is about 1/10 degree in this example. Since it is desired to detect the desired play between actuator and object (as described below), this desired play should not be less than the sensing resolution of sensor (e.g., 1/10 degree). Preferably, the desired play between actuator and object would be at least ⅕ degree in this example, since the encoder could then detect two pulses of movement, which would provide a more reliable measurement and allow the direction of the movement to be more easily determined.

The sensor should also be coupled to the shaft as tightly as possible so that the sensor can detect the desired play of axis and object. Any play between sensor and object should be minimized so that such play does not adversely affect the sensor's measurements. Typically, any inherent play between sensor and object should be less than the sensing resolution of the sensor, and preferably at least an order of magnitude less than the preferred sensing resolution. Thus, in the example above, the play between sensor and object should be less than 1/10 degree and preferably less than 1/100 degree. Use of steel or other rigid materials for axes and other components, which is preferred, can allow the play between sensor and object to be made practically negligible for purposes of the present invention.

Host computer and microprocessors are well known in the art and are commercially available. Such computers typically have standard interfaces which include a serial port or additional hardware which allow faster signal processing.

One embodiment of the current invention has 7 degrees-of-freedom for position feedback, of which, four degrees of freedom also provide force feedback. The haptic interface acts as a master controller for the robotic arm and laparoscopic tool or other similar tools, attached at the end of the robotic arm. To provide the user with force feedback during a typical tissue manipulation task, a force feedback mechanism is incorporated in the design of device whereby force feedback is along the X, Y, and Z-axes. Additionally the grasping and parting tasks have an incorporated force feedback mechanism. Similarly, other standard features of haptic mechanisms such as backdriveability, low friction, high transparency, adequate force ranges, static balancing, and a large workspace were incorporated into the design.

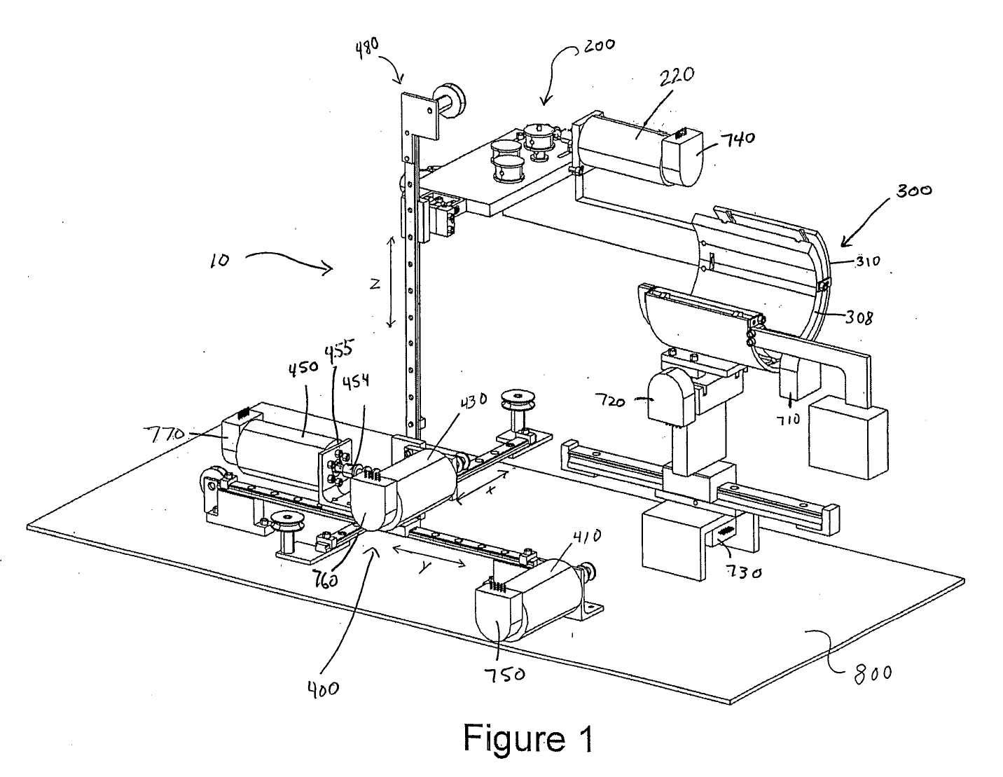

These and other advantages of the present invention will become apparent to those skilled in the art upon a reading of the following specification of the invention and a study of the several figures of the drawing. Reference will now be made in detail to the preferred aspects of the invention, and an example of which is illustrated in the accompanying drawings. An exemplary embodiment of the haptic interface of the present invention is shown in FIGS. 1-4 and is designated generally by the reference number 10.

As shown in FIGS. 1-4, the haptic interface 10 is a closed kinematic chain that consists of a hand and forearm rest 300, a grasper assembly 200 for two fingers, such as a thumb and index finger for example, and a decoupled 3 degrees-of-freedom spatial force feedback mechanism 400. The hand and forearm rest contains 4 degrees-of-freedom for positioning the user's arm, i.e. roll, pitch, yaw, of the wrist and linear motion for the forearm. This creates an ergonomic platform that conforms to the natural motions of the human arm for typical manual manipulation tasks.

The grasping/parting mechanism is coupled to the direct drive DC motor that allows for grasping/parting tasks using two fingers of the user, such as the thumb and index finger. This mechanism enables full controllability of a grasping/parting mechanism such as the laparoscopic tool's manipulation on the robot's end effector. Therefore, the user can control the angle of the jaws of the laparoscopic tool and also receive force feedback through the DC motor as detected by sensors in the laparoscopic tool. The decoupled spatial force feedback mechanism consists of a 3 degrees-of-freedom positioning stage that is attached to the hand and forearm rest at the grasping/parting mechanism through a universal joint. The force feedback mechanism was designed to apply all forces to the user at the grasping/parting mechanism rather than through the joints of the hand and forearm rest. This enhances the transparency of the haptic interface by providing feedback, which is more analogous to conventional open surgery where the surgeon primarily receives feedback at the point of contact with the soft tissue and/or organs.

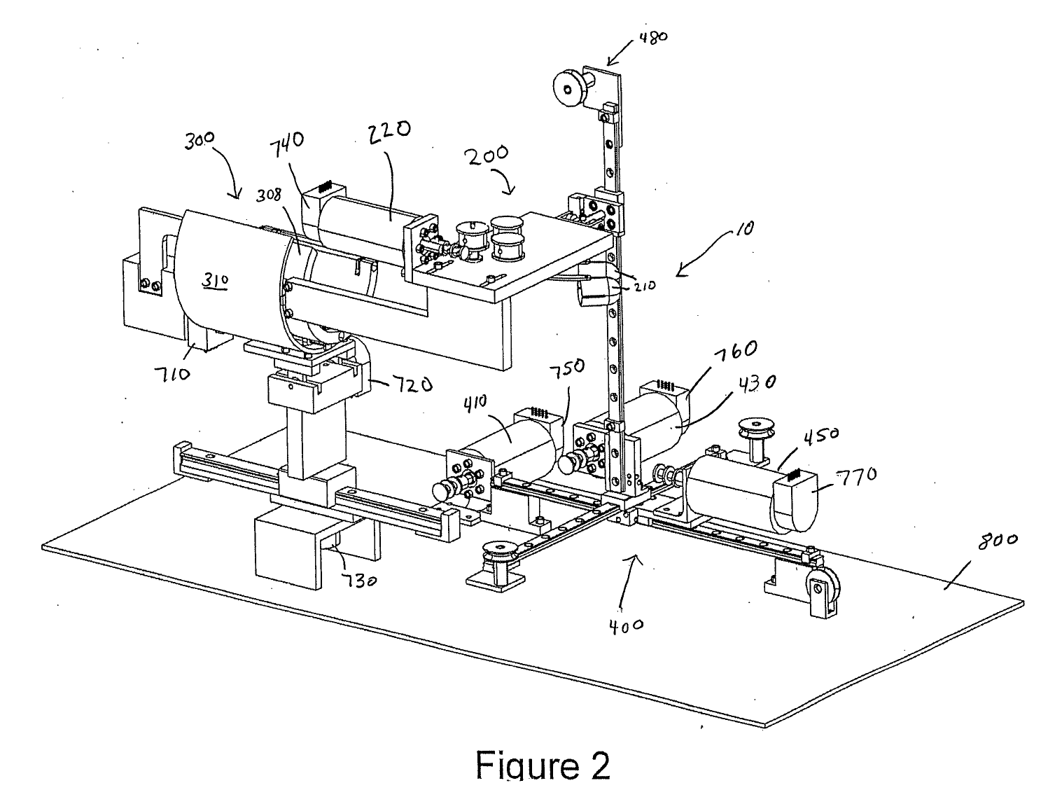

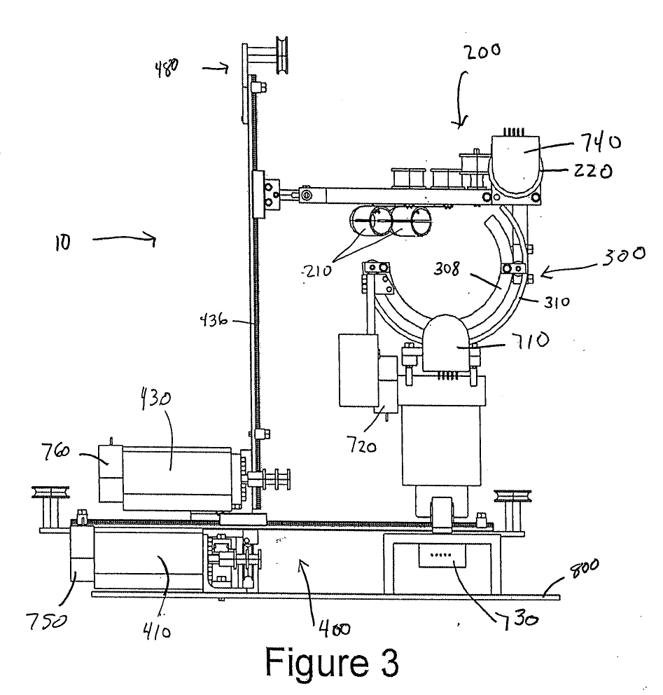

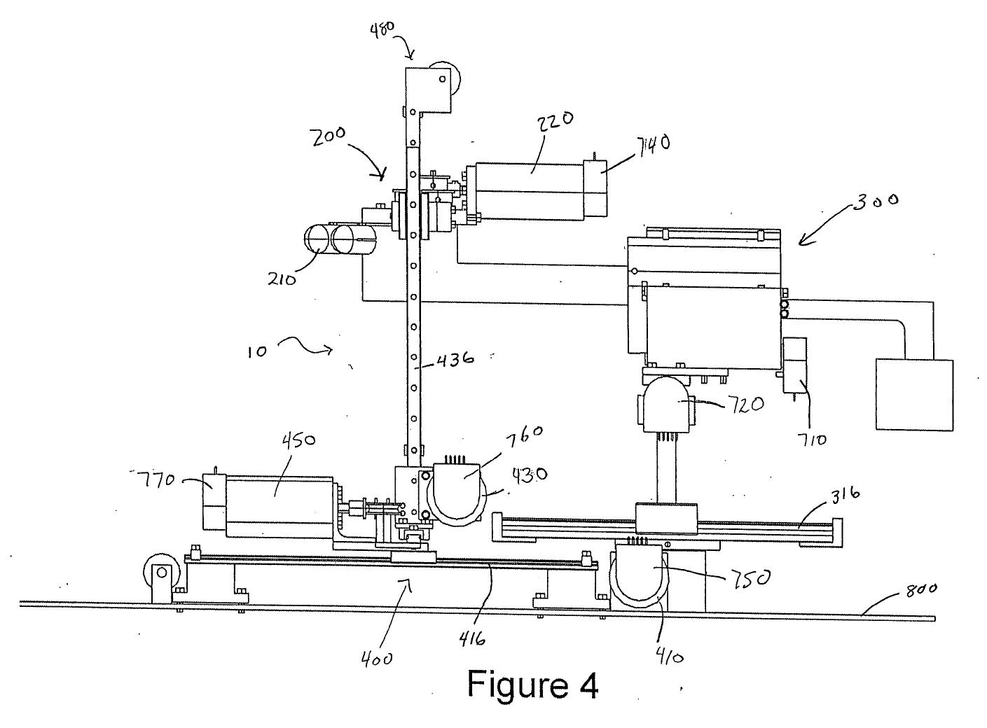

FIG. 1 represents a three-dimensional view of the haptic interface 10. The major components are a grasping/parting assembly 200, a hand and forearm rest 300, a spatial force feedback mechanism 400, encoders 710, 720, 730, 740, 750, 760, and 770, and a mounting platform 800. FIG. 2 represents a 3-dimensional view of the back of the haptic interface 10 shown in FIG. 1. The back view shows grasping/parting assembly 200 with thimbles 210, hand and forearm rest 300, spatial force feedback mechanism 400, encoders 710, 720, 730, 740, 750, 760, and 770, and mounting platform 800. FIGS. 3-4 show other views of the same haptic interface 10. FIG. 3 is a front view of the complete haptic assembly while FIG. 4 is a side view of the complete haptic assembly 10.

The hand and forearm rest 300 contains four degrees-of-freedom as determined by the natural motion of the human hand. The orientations of the wrist, elbow and shoulder joints are translated to four degrees-of-freedom in laparoscopic surgery (three rotational and one translational) due to the pivot at the incision point. Therefore, to effectively map the position and orientation of the laparoscopic tool in the robot-assisted surgical system, the map must detail the four degrees-of-freedom of the laparoscopic tool to the user.

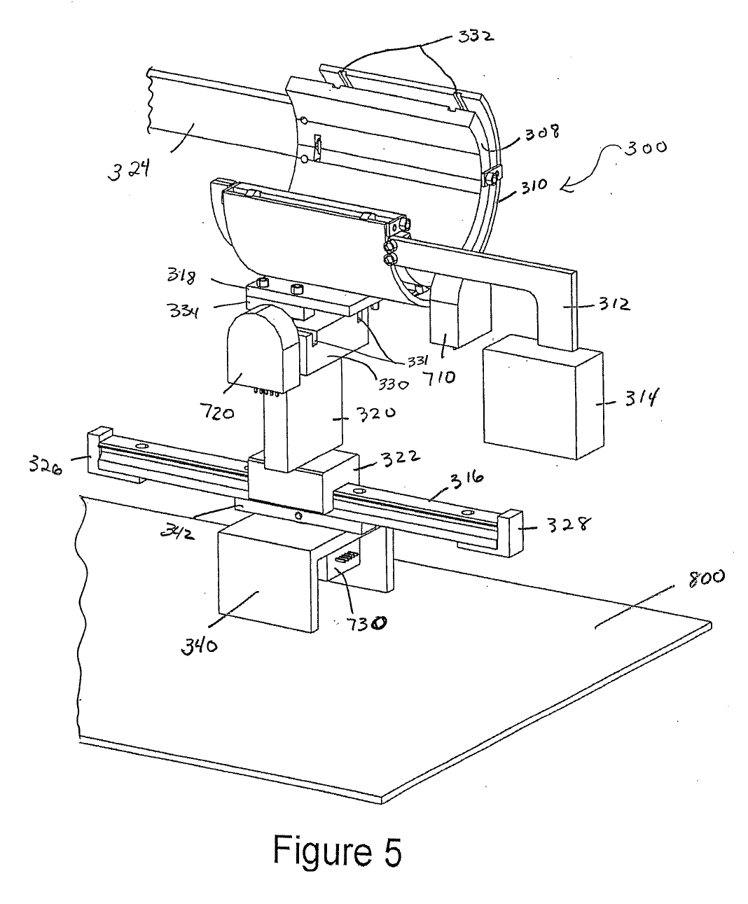

A schematic of the hand/forearm rest 300 is shown in FIG. 5. The hand/forearm rest 300 consists of a base mounting bracket 340. The mounting bracket 340 has two parallel vertical rising plates and a horizontal plate. An encoder 730 is mounted on the underside of the mounting bracket 340. Encoder 730 measures the yaw motion of the hand/forearm rest 300. A linear slide bar 316 is mounted to this mounting bracket 340 by a linear slide mount plate 342 which allows rotation about the vertical axis. On the top side of the linear slide bar 316, linear slide mounting bracket 322 is mounted. Linear slide mounting bracket 322 provides approximately six inches of motion in the global x-y plane. The linear slide bar 316 has stops 326 and 328 attached to limit the motion of the linear slide mounting bracket 322. The linear slide mounting bracket 322 has attached to it a vertical mount plate 320 and a third mounting plate 330. Third mounting plate 330 includes an adjustable means provided by grooves 331 with a means such as bearings (not shown) to attach to the fourth mounting plate 318 via mounting bracket 334. Encoder 720 attaches to the mounting plate 330 with its shaft connected to a bearing (not shown) inside mounting bracket 334. The mounting bracket 334 is mounted for rotational movement relative to the third mounting plate 330 to allow for the outer forearm rest arc 310 to pitch.

The hand/forearm rest 300 consists of an inner forearm rest arc 308 and an outer forearm rest arc 310. The encoder 720 detects the pitch motion of the hand/forearm rest 300. The outer forearm rest arc 310 includes means to attach a counterweight arm 312, which in turn has a counterweight 314 attached thereto. Between the inner forearm rest arc 308 and the outer forearm rest arc 310, is a means such as bearings (not shown) for allowing the inner forearm rest arc 308 to rotate relative to the outer forearm rest arc 310. The rotational means consists of a bearing assembly mounted in grooves 332. Rotation of the forearm creates the roll of the mechanism by causing inner forearm rest arc 308 to roll relative to outer forearm rest arc 310. Encoder 710 is attached to inner forearm rest arc 308 to encode the roll. The outer forearm rest arc 310 also has a link 324 to the grasping/parting assembly 200.

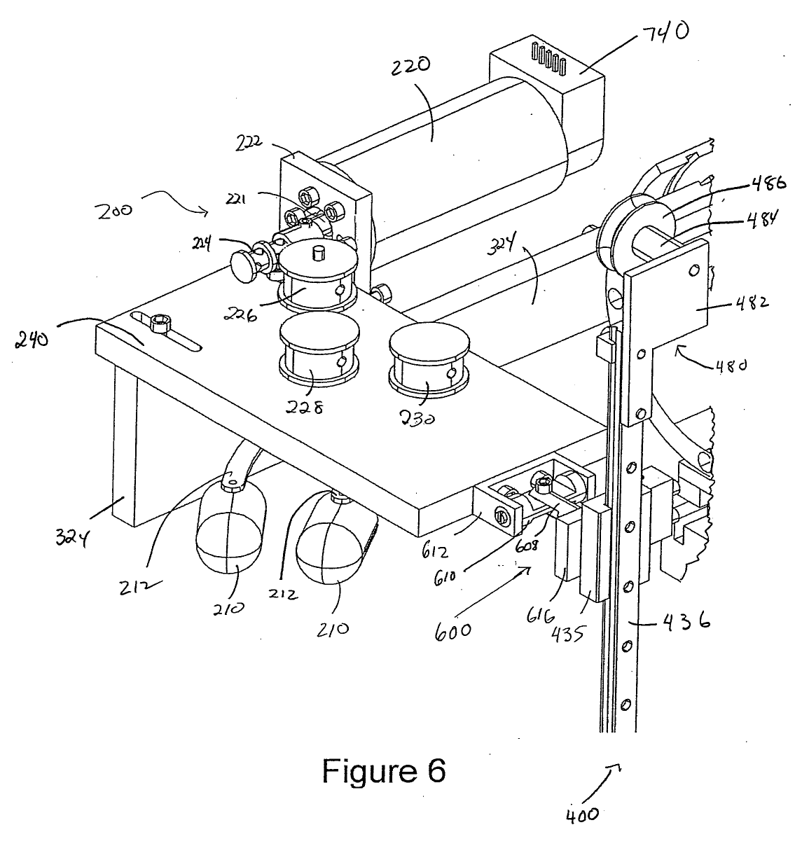

Turning now to FIG. 6, which is a schematic of the grasping/parting assembly 200 (top view). The grasper assembly contains a capstan 224, thimble assemblies 210 and a grasper motor 220 for actuation of the thimble assemblies 210. The grasper motor 220 attaches to the grasping/parting assembly 200 by means of a mounting plate 222, which in turn attaches to the platform 240. Encoder 740 attaches to the grasper motor 220 opposite of capstan 224 and measures the motion of the thimble assemblies 210. Platform 240 attaches to the forearm link 324. Thimble pulleys 228 and 230 are attached to the platform 240 using ball bearings. Each thimble pulley 228, 230 connects to a linkage 212 on the underside of the platform 240. These linkages 212 then extend approximately 3 inches and each has a thimble 210 attached. By using the linkages 212, the natural motion of the finger and thumb is used, for example, during grasping/parting tasks and an ergonomic mechanism is provided for the user's hand.

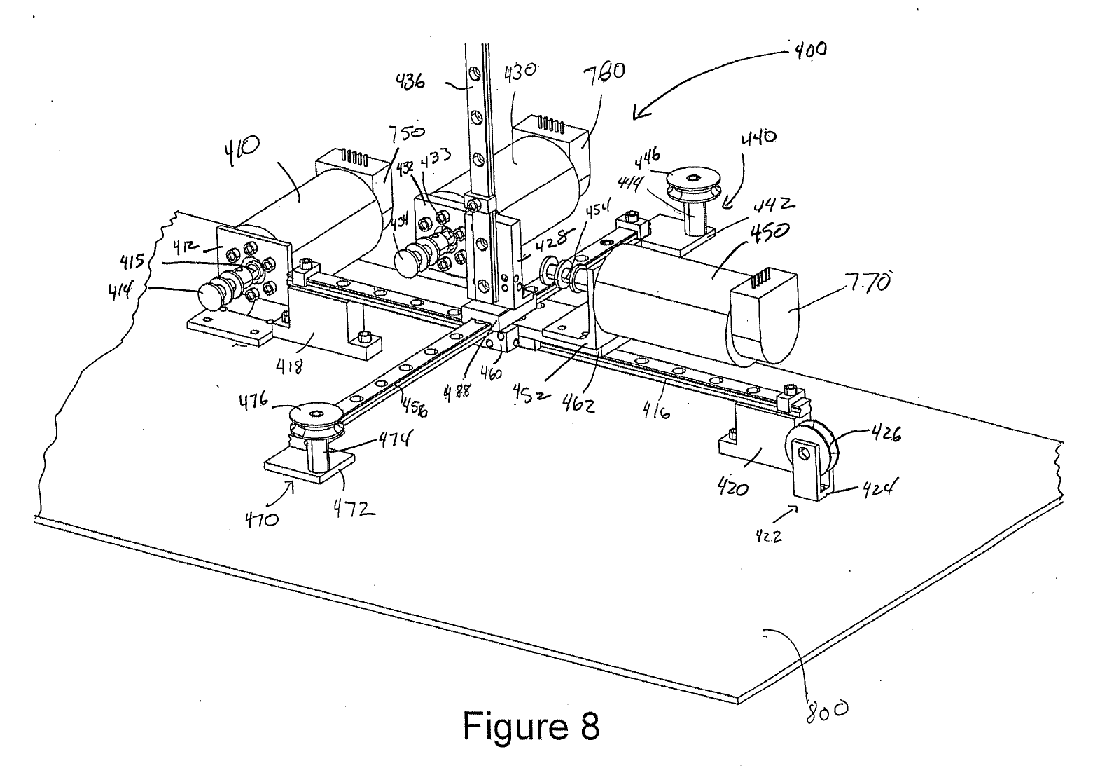

For actuation of the grasping/parting mechanism 200, steel cables (not shown) attached to capstan 224 are used to rotate the intermediate pulley 226, which increases the torque from capstan 224 in a 3:1 ratio. A second steel cable then travels from the intermediate pulley 226 to the thimble pulley 228 with another torque increase in a 3:1 ratio, and a third steel cable travels from thimble pulley 228 to thimble pulley 230 in a lemniscate pattern. Therefore, the two thimble pulleys 228, 230 rotate in opposite directions and create the grasping/parting motion of the two thimbles 210. Hence, this assembly has the capability to provide force feedback to the user for both grasping and parting tasks. An intermediate pulley 226 is mounted on the top side of the platform 240 using ball bearings. The platform 240 also has a means 612 to attach the grasping/parting assembly 200 to the spatial force feed back mechanism 400 through a universal joint 600. The spatial force feedback mechanism 400 is described in greater detail with reference to FIG. 8.

The grasping/parting assembly 200 consists of a grasper DC motor 220, pulleys 226, 228, 230, encoder 740, and thimble assemblies 210, 212 that allow the user of the haptic interface to control grasping objects with two fingers such as the thumb and index finger, for example. The grasper motor 220 is attached to the platform 240 via mounting plate 222 with capstan 224 attached to a shaft 221 of grasper motor 220.

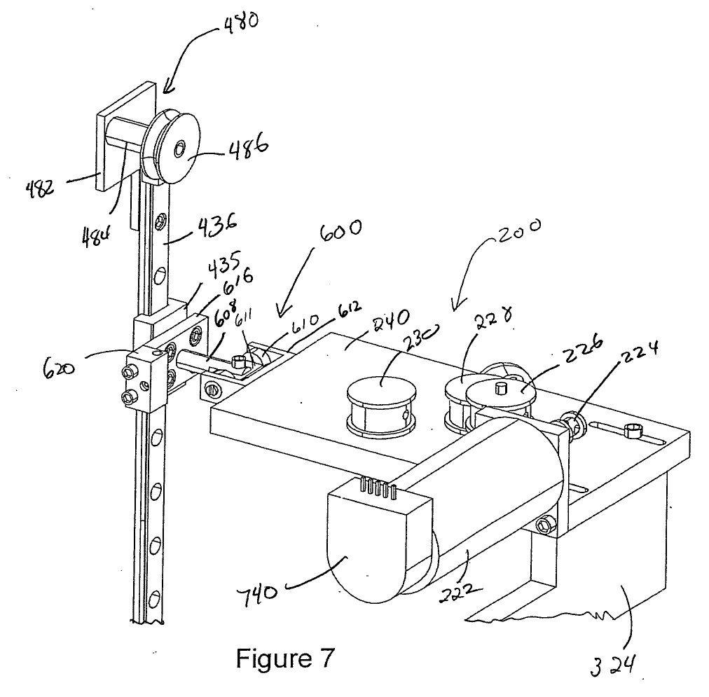

FIG. 7, shows universal joint 600. Mounting means 612 holds a rotatable rod unit 610. Rotatable rod unit 610 has a pair of upper and lower slots 611, for attaching a spacer rod 608 to mounting plate 616. Mounting plate 616 is attached to the Z direction slide mount 435. The universal joint 600 attaches the grasping/parting assembly 200 to the Z direction slide mount 435.

FIG. 8 is a detailed view of the spatial force feedback mechanism 400. Y motor 410 is mounted to platform 800 by mounting bracket 412. Y motor 410 has a capstan 414 mounted to the motor shaft 415. Y motor 410 has encoder 750 mounted opposite of capstan 414 to measure the rotation of the Y motor shaft 415. Y motor 410 is mounted at one end of a Y directional linear slide bar 416. The Y direction linear slide bar 416 is mounted on the platform 800 by mounting brackets 418 and 420. Mounted at the distal edge of the mounting bracket 420 is a Y pulley assembly 422 consisting of a mounting bracket 424 and a vertically oriented Y motor pulley 426. A steel cable (not shown) will be attached to Y motor capstan 414 to run from the Y motor capstan 414 through the Y pulley 426 and back to the Y motor capstan 414. A section of the steel cable will be attached to the Y direction cable mount 460 to move the X and Z direction linear slide bars 436, 456 in the Y direction. X motor 450 is mounted on top of the Y direction linear slide mounting bracket 462 by a mounting bracket 452. A capstan 454 is mounted to the shaft 455 of the X motor 450. Encoder 770 is mounted on X motor 450 opposite of capstan 454 to measure the rotation of the X motor shaft 455 (see FIG. 1). X direction linear slide bar 456 is mounted to mounting bracket 462. An X direction linear slide 488 is slide mounted for movement in the X direction on the X direction linear slide bar 456. At each end of the X direction linear slide bar 456 is attached an X direction pulley assembly 440 and 470. Pulley assembly 440 consists of a mounting bracket 442, a vertical spindle 444 and a horizontal pulley 446. Pulley assembly 470 consists of a mounting bracket 472, a vertical spindle 474 and a horizontal pulley 476.

Attached to the X direction slide 488 is a Z direction slide bar mounting bracket 428. The Z direction slide bar mounting bracket 428 is attached to the Z direction slide bar 436. The Z direction slide bar 436 also has a means at the bottom end to vertically mount the Z direction slide bar 436 to the X direction slide 488. A steel cable will attach the X capstan 454 to the X direction pulleys 446, 476. A section of the same steel cable is attached to the Z direction slide bar mounting bracket 428 to move the Z direction linear slide bar 436 in the X direction. Z motor 430 is attached to Z motor mounting bracket 432. Z motor 430 has a capstan 434 attached to the shaft 433 of Z motor 430. Z motor 430 has encoder 760 mounted opposite of capstan 434 to measure the rotation of the Z motor shaft 433. Z motor 430 is attached to the Z direction slide bar mounting bracket 428 by Z motor mounting bracket 432. Referring now to FIG. 6, Z direction linear slide bar 436 is shown with the Z direction pulley assembly 480. Z direction pulley assembly 480 consists of the pulley assembly mounting bracket 482, a horizontal shaft 484 and a vertical pulley 486. One end of a steel cable will be attached to the Z motor 430 at the capstan 434, travel around the Z direction pulley 486 and back to the Z motor capstan 434. The steel cable will be also attached to mounting plate 616 via Z direction cable mount 620 (shown in FIG. 7).

The preferred embodiment uses multiple back-drivable non-geared brushless DC motors with rotational sensing to effect and control the superposition of translational displacement with force application and angular displacement with torque, thus providing arbitrary, programmed application of forces, torques, and displacements to the handle in any direction. The brushless motor commutation can be accomplished using encoder position readings.

Thus a haptic interface for use in manipulation tasks has been described. This interface can be used for a variety of applications such as robotically-assisted minimally invasive surgery, automotive industry, gaming industry, rehabilitation aid for people with finger, hand, and/or forearm injuries, etc. It has four degrees-of-freedom of force feedback that includes one for grasping/parting force feedback and three for spatial force feedback. The spatial force feedback uses prismatic joints to create force feedback along three independent orthogonal coordinate axes. A net of three additional passive joints for positional feedback allows for a total of seven degrees-of-freedom positional feedback for the user interface. The present invention provides for a better ergonomic design that is more user friendly. The present invention also provides for a larger force feedback capability as well as providing for a larger workspace for the given range of the force feedback capability. Additionally, the present invention has a single independent motor for each of the X, Y and Z direction force feedback axes and the grasping/parting assembly. Finally, the present invention provides for an intuitive haptic interface for spatial manipulation.

The present invention is designed as non-portable haptic interface, which allows the use of higher power actuators, as the user does not carry the complete weight of the spatial (X, Y, and Z directions) actuators but would feel some inertia of the overall device. The higher-powered actuator allows for an increased capability of force feedback for the grasping and parting forces. The single independent motor for each of the X, Y and Z direction force feedback axes combined with a single independent motor for the grasping and parting forces represents an improvement over the prior art.

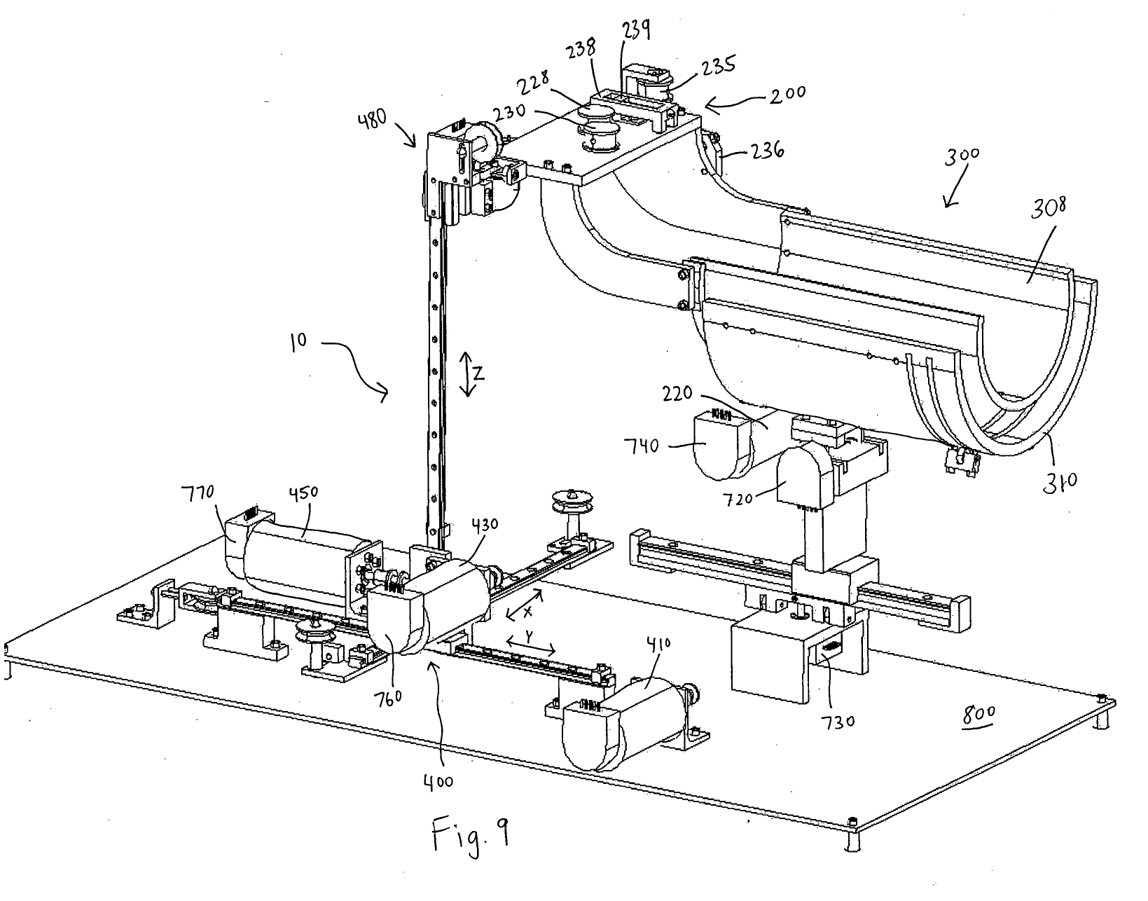

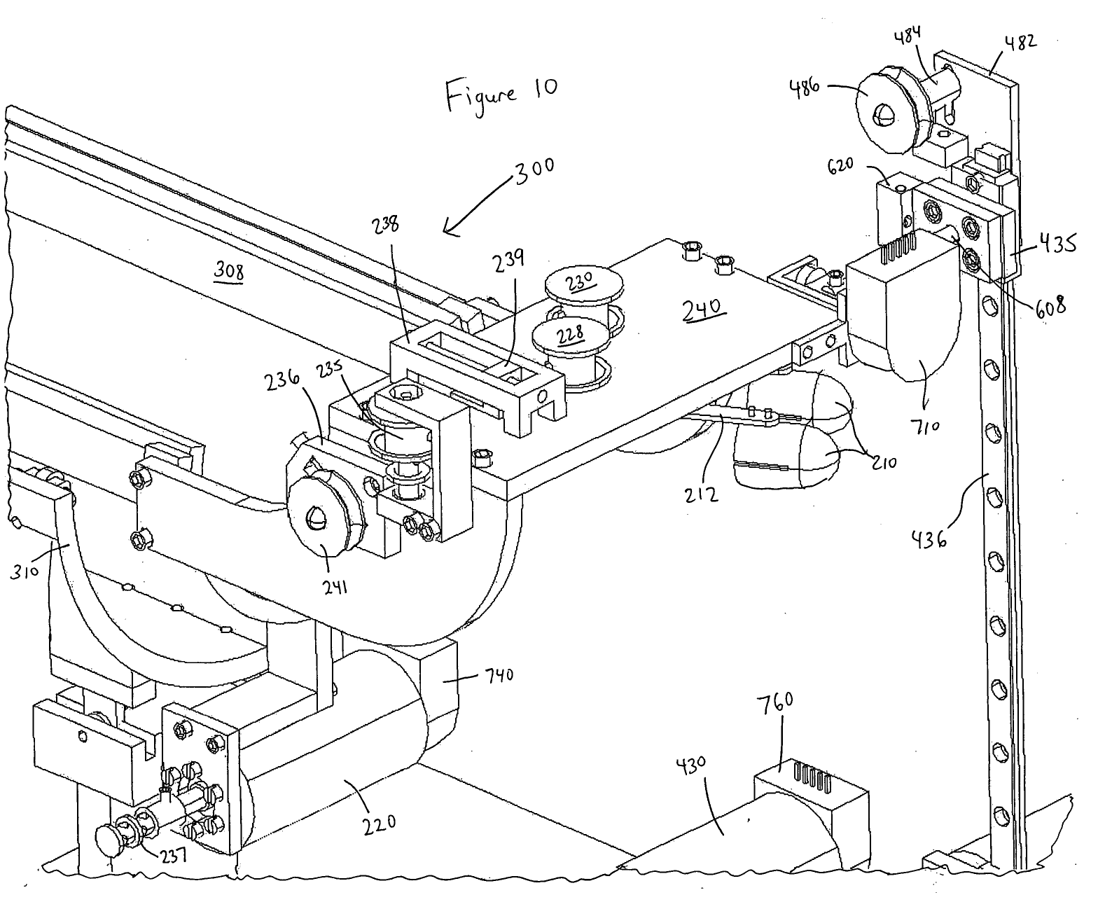

FIGS. 9-10 show an alternative embodiment of a haptic interface 10 that has a mechanism for increasing the torque to thimbles 210. Grasping/parting mechanism 200 consists of a direct drive motor 220, pulleys 228 and 230, and thimble assembly 210 that allow the user of haptic interface 10 to control grasping/parting of objects with two fingers, such as the thumb and index finger, for example. The direct drive motor 220 with encoder 740 and capstan 237 is attached to and mounted below the forearm rest 300 to minimize interference with the user's arm. A two-stage pulley system utilizing steel cables is used to transmit motion from motor 220 to thimbles 210 via an intermediate pulley 235. A steel cable travels from capstan 237 through a tensioner 236 and then to the intermediate pulley 235, which is mounted to the side of the grasping/parting mechanism 200. The intermediate pulley 235 increases the torque from capstan 237 by, for example, a factor of three. Tensioner 236 includes an adjustable idle pulley 241 to provide sufficient tensioning of the steel cable.

The second stage of the transmission uses a second steel cable running from intermediate pulley 235, through tensioner 238, and then to the thimble pulleys 230 and 228, for each finger thimble 210. The cable is wound in different directions on each thimble pulley 228, 230, which allows for equal and opposite rotation of thimbles 228, 230 for opening/closing motions. Hence, this assembly has the capability to provide force feedback to the user for both grasping and parting tasks. Tensioner 238, for the second stage, includes an adjustable idle pulley (not shown) inside idle pulley housing 239. Between intermediate pulley 235 and thimble pulleys 228, 230, there is also a torque increase, for example, by a factor of three, that results in a total increase of the torque from capstan 237 to thimble pulleys 228, 230, for example, by a factor of nine.

While this invention has been described in terms of a preferred embodiment, it is contemplated that alterations, modifications and permutations thereof will become apparent to those skilled in the art upon a reading of the specification and study of the drawings. Furthermore, certain terminology has been used for the purposes of descriptive clarity, and not to limit the present invention.

### REFERENCES

[1] A. R. Lanfranco, A. E. Castellanos, J. P. Desai, and W. Meyers, “Robotic Surgery: A Current Perspective,” Annals of Surgery, pp. In Press, 2003.
[2] A. Bicchi, G. Canepa, D. DeRossi, P. lacconi, and E. Scilingo, “A sensor-based minimally invasive surgery tool for detecting tissue elastic properties,” IEEE International Conference on Robotics and Automation, 1996.
[3] J. Dargahi, M. Parameswaran, and S. Payandeh, “A Micromachined Piezoelectric Tactile Sensor for an Endoscopic Grasper—Theory, Fabrication and Experiments,” Journal of Microelectromechanical Systems, vol. 9, pp. 329-335, 2000.
[4] V. V. H. t. Dingshoft, M. Lazeroms, A. v. d. Ham, W. Jongkind, and G. Hondred, “Force reflection for a laparoscopic forceps,” 18th Annual International Conference of the IEEE Engineering in Medicine and Biology Society, 1996.
[5] T. Hu, A. E. Castellanos, G. Tholey, and J. P. Desai, “Real-Time Haptic feedback in Laparoscopic tool for use in Gastro-intestinal Surgery,” Fifth International Conference on Medical Image Computing and Computer Assisted Intervention (MICCAI), 2002.
[6] A. Krupa, G. Morel, and M. D. Mathelin, “Achieving high precision laparoscopic manipulation through adaptive force control,” IEEE International Conference on Robotics and Automation, 2002.
[7] A. J. Madhani, G. Niemeyer, and J. K. Salisbury, “The Black Falcon: A Teleoperated Surgical Instrument for Minimally Invasive Surgery,” IEEE/RSJ International Conference on Intelligent Robotic Systems, vol. 2, pp. 936-944, 1998.
[8] V. F. Munoz, C. Vara-Thorbeck, J. G. DeGabriel, J. F. Lozano, E. Sanchez-Badajoz, A. Garcia-Cerezo, R. Toscano, and A. Jimenez-Garrido, “A medical robotic assistant for minimally invasive surgery,” IEEE International Conference on Robotics and Automation, 2000.
[9] D. T. V. Pawluk, J. S. Son, P. S. Wellman, W. J. Peine, and R. D. Howe, “A Distributed Pressure Sensor for Biomechanical Measurements,” ASME Journal of Biomechanical Engineering, vol. 102, pp. 302-305, 1998.
[10] J. Rosen, B. Hannaford, M. MacFarlane, and M. Sinanan, “Force Controlled and Teleoperated Endoscopic Grasper for Minimally Invasive Surgery—Experimental Performance Evaluation,” IEEE Transactions on Biomedical Engineering, vol. 46, pp. 1212-1221, 1999.
[11] D. Salle, F. Gosselin, P. Bidaud, and P. Gravez, “Analysis of Haptic Feedback Performances in Telesurgery Robotic Systems,” IEEE International Workshop on Robot and Human Interactive Communication, 2001.
[12] E. Scilingo, D. DeRossi, A. Bicchi, and P. lacconi, “Sensor and devices to enhance the performance of a minimally invasive surgery tool for replicating surgeon's haptic perception of the manipulated tissues,” IEEE International Conference on Engineering in Medicine and Biology, 1997.
[13] G. Tholey, A. Pillarisetti, W. Green, and J. P. Desai, “Design, Development, and Testing of an Automated Laparoscopic Grasper with 3-D Force Measurement Capability,” Second International Symposium on Medical Simulation Emerging Science Enabling Technologies, Boston, Mass., 2004.
[14] M. Tavakoli, R. V. Patel, and M. Moallem, “A Forced Reflective Master-Slave System for Minimally Invasive Surgery,” IEEE International Conference on Intelligent Robots and Systems, Las Vegas, Nev., 2003.
[15] R. H. Taylor, J. Funda, B. Eldridge, S. Gomery, K. Gruben, D. LaRose, M. Talamini, L. Kavoussi, and J. Anderson, “A telerobotic assistant for laparoscopic surgery,” IEEE Engineering in Medicine and Biology, vol. 14, pp. 279-286, 1995.
[16] T. H. Massie and K. J. Salisbury, “Force reflecting haptic interface.” US: Massachusetts Institute of Technology, 1993.
[17] M. Ueberle and M. Buss, “Design, Control, and Evaluation of a New 6 DOF Haptic Device,” IEEE International Conference on Intelligent Robots and Systems, Lausanne, Switzerland, 2002.
[18] E. L. Faulring, J. E. Colgate, and M. A. Peshkin, “A High Performance 6-DOF Haptic Cobot,” IEEE International Conference on Robotics and Automation, New Orleans, La., 2004.
[19] K. Kim, W. K. Chung, andY. Yourn, “Design and Analysis of a New 7-DOF Parallel Type Haptic Device: PATHOS-II,” IEEE International Conference on Intelligent Robots and Systems, Las Vegas, Nev., 2003.
[20] L. Birglen, C. Gosselin, N. Pouliot, B. Monsarrat, and T. Laliberte, “SHaDe, A New 3-DOF Haptic Device,” IEEE Transactions on Robotics and Automation, vol. 18, pp. 166-175, 2002.
[21] J. Yoon and J. Ryu, “Design and Analysis of a New Haptic Device Using a Parallel Mechanism,” IEEE International Conference on Intelligent Robots and Systems, 2000.
[22] R. E. Ellis, O. M. Ismaeil, and M. G. Lipsett, “Design and Evaluation of a High-Performance Haptic Interface,” Robotica, vol. 14, pp. 321-327, 1996.
[23] P. A. Millman and J. E. Colgate, “Design of a Four Degree-of-Freedom Force-Reflecting Manipulandum with a Specified Force/Torque Workspace,” IEEE International Conference on Robotics and Automation, Sacramento, Calif., 1991.
[24] S. S. Lee and J. M. Lee, “Design of a general purpose 6-DOF haptic interface,” Mechatronics, vol. 13, pp. 697-722, 2003.
[25] J. H. Lee, K. S. Eom, B. J. Yi, and I. H. Suh, “Design of a New 6-DOF Parallel Haptic Device,” IEEE International Conference on Robotics and Automation, Seoul, Korea, 2001.
[26] T. Koyama, I. Yamano, K. Takemura, and T. Maeno, “Multi-Fingered Exoskeleton Haptic Device using Passive Force Feedback for Dexterous Teleoperation,” IEEE International Conference on Intelligent Robots and Systems, Lausanne, Switzerland, 2002.
[27] W. Chou, T. Wang, and J. Xiao, “Haptic Interaction with Virtual Environment Using an Arm Type Exoskeleton Device,” IEEE International Conference on Robotics and Automation, New Orleans, La, 2004.
[28] B. H. Choi and H. R. Choi, “A Semi-direct Drive Hand Exoskeleton Using Ultrasonic Motor,” IEEE International Workshop on Robot and Human Interaction, Pisa, Italy, 1999.
[29] K. Vlachos, E. Papadopoulos, and D. N. Mitropoulos, “Design and Implementation of a Haptic Device for Training in Urological Operations,” IEEE Transactions on Robotics and Automation, vol. 19, pp. 801-809, 2003.
[30] R. Baumann, W. Maeder, D. Glauser, and R. Clavel, “The Pantoscope: A Spherical Remote-Center-of-Motion Parallel Manipulator for Force Reflection,” IEEE International Conference on Robotics and Automation, Albuquerque, N. Mex., 1997.
[31] S. Payandeh and T. Li, “Towards new designs of haptic devices for minimally invasive surgery,” International Congress Series, pp. 775-781, 2003.
[32] V. Hayward, P. Gregorio, O. Astley, S. Greenish, and M. Doyon, “Freedom-7: A High Fidelity Seven Axis Haptic Device With Application to Surgical Training,” Experimental Robotics Vi, pp. 445-456, 1998.
[33] E. Papadopoulos, K. Vlachos, and D. Mitropoulos, “Design of a 5-d of Haptic Simulator for Urological Operations,” IEEE International Conference on Robotics and Automation, Washington, DC, 2002.

What is claimed is:

1. A haptic device which comprises:
a spatial mechanism including at least one rotational joint and at least one linear joint,
a universal joint connected to said spatial mechanism;
a user interface including at least one rotational joint, at least one linear joint, and a grasping/parting mechanism connected to said universal joint;
a plurality of sensors configured to measure forces on the joints of said spatial mechanism and generate sensor signals representative of said forces;
a plurality of actuators coupled to said sensors to receive said sensor signals, and operatively coupled to the grasping/parting mechanism to produce a haptic effect to the user responsive to the sensor signals.

2. The apparatus of claim 1, where the user interface further comprises a hand, wrist, and arm rest.

3. The apparatus of claim 1, wherein the grasping/parting mechanism comprises at least two finger rests.

4. The apparatus of claim 1, wherein the haptic effect provides force feedback to the user via the grasping/parting mechanism.

5. The apparatus of claim 1, wherein the haptic device provides four degrees of force feedback to the user.

6. The apparatus of claim 1, wherein the user interface permits seven degrees of position feedback.

7. The apparatus of claim 3, wherein the grasping/parting mechanism further comprises a motor.

8. The apparatus of claim 5, wherein the spatial force feedback mechanism comprises at least three motors and at least one encoder operatively associated with each said motor.

9. The apparatus of claim 8, wherein the spatial force feedback mechanism provides force feedback in three orthogonal directions.

10. The apparatus of claim 1, wherein the user interface comprises three rotational joints and said rotational joints provide pitch, yaw and roll motion.

11. The apparatus of claim 10, wherein said user interface further comprises an arm rest operatively connected to said three rotational joints and each said rotational joint has an encoder operatively associated therewith.

12. A method for providing haptic feedback to a user comprising the steps of:
providing a spatial mechanism having three degrees of freedom,
sensing forces exerted on said spatial mechanism for each degree of freedom, and
translating said sensed forces into forces exerted in three degrees of freedom on a universal joint connected to a grasping/parting mechanism suitable for grasping by a thumb and index finger of the user.

13. A method as claimed in claim 12, wherein said haptic feedback comprises grasping and parting force feedback.

14. A method as claimed in claim 13, wherein said haptic feedback comprises four degrees of freedom force feedback.

15. A method as claimed in claim 14, further comprising the steps of:
providing a user interface capable of pitch, yaw and roll motion, and
manipulating said user interface to cause movement in said spatial mechanism.

16. A method as claimed in claim 15, wherein said spatial mechanism is associated with a device in a manner whereby manipulation of said user interface causes manipulation of said device by said spatial mechanism.

17. A method as claimed in claim 16, wherein said device can be manipulated in at least three directions corresponding to the pitch, yaw and roll motions of said user interface.

18. A method as claimed in claim 17, wherein said device can be manipulated to cause a grasping/parting motion of said device by manipulation of said grasping/parting mechanism.
